(19) United States
(12) Patent Application Publication
(10) Pub. No.: US 2006/0025797 A1
(43) Pub. Date: Feb. 2, 2006

(54) Cannula for in utero surgery

(76) Inventors: James Lock, Boston, MA (US); Paul C. Kierce, Woburn, MA (US); Audrey C. Marshall, Boston, MA (US)

Correspondence Address: Amy E. Rinaldo;KOHN & ASSOCIATES, PLLC, 30500 Northwestern Highway, Suite 410, Farmington Hills, MI, 48334, US

(21) Appl. No.: 11/182,928

(22) Filed: Jul. 15, 2005

Related U.S. Application Data
(63) Continuation-in-part of application No. 10/891,937, filed on Jul. 15, 2004.

(51) Int. Cl. A61M 29/00

(52) U.S. Cl. 606/191

(57) ABSTRACT

A cannula, a straightener, and a cannula and straightener combination for insertion into tissue, the cannula including an elongated rigid hollow tube having a proximal end, a distal end, and a passageway extending therebetween. The distal end includes a memory of directionality to bend about a radius. The cannula straightener straightens the distal end of the cannula when inserted through the passageway of the cannula. The present invention also includes methods of inserting a cannula, removing a cannula, and performing surgery with a cannula in tissue or in fetal tissue. The present invention further includes methods of performing biliary cannulation in a transhepatic approach, performing fetal aortic valvuloplasty, placing catheters into the brachial plexus for pain management, placing catheters into the epidural space for anesthesia, thoracic dissection treatment, laparoscopic dissection, and hydro dissection of tissue.

### CROSS-REFERENCE TO RELATED APPLICATIONS

This CIP patent application claims priority to U.S. patent application Ser. No. 10/891,937, filed Jul. 15, 2004, which is incorporated herein by reference.

### BACKGROUND OF THE INVENTION

1. Technical Field

The present invention relates to a medical device for insertion into tissue during surgical procedures. In particular, the present invention relates to cannulas, trocars, obturators, and uses thereof.

2. Description of the Related Art

There are many minimally invasive surgical procedures wherein surgery is performed without making a large incision in a patient. Patients of these processes benefit by receiving less trauma to the body and save money by reduced hospitalization time and reduced therapy time. Such minimally invasive procedures range from cardiovascular, spinal, laparoscopic, thoracoscopic, and various anesthesia procedures. Minimally invasive surgery is also commonly known as endoscopic surgery because an endoscope is inserted to view the inside of the body so that the physicians can monitor the path of their instruments.

During a surgical procedure, a cannula mounted coaxially on a sharp-pointed trocar or blunt pointed obturator is commonly used to percutaneously access vessels and internal structures. The point on the trocar or obturator is used to puncture through surrounding structures and tissues to lead the cannula to the vessel or structure of interest. Once the tip of the trocar/obturator and the tip of the cannula are in the structure of interest, the trocar/obturator is removed and the cannula remains. The lumen of the cannula, previously occupied by the trocar/obturator, can then be used to introduce or deliver various items such as pharmaceuticals, diagnostic or therapeutic devices, implantables, and instruments into the vessel or structure to perform the needed surgery or procedure.

In order to perform balloon dilation procedures in fetuses, one must be able to insert and remove a balloon dilation catheter at the structure of interest. These catheters are straight and blunt, with a very flexible shaft and an irregular profile, particularly after inflation/deflation. For this reason, the balloon is introduced and removed through an introducer cannula.

Fetal cardiac interventions, however, demand specific qualities of the trocar/obturator and introducer cannula. The combination of the trocar/obturator and cannula must be straight and inflexible through a length of 10-15 cm in order to have exact control of the tip of the trocar/obturator as it is advanced through maternal and fetal tissue to the target structure. Performing cardiac surgery on a fetus can require placement of wires or catheters at an angle different from the angle of straight access. The cannula must remain inflexible after the trocar/obturator has been removed to allow precise tip control.

There are many other procedures where, in order to gain access to a distant vascular or nonvascular chamber, the operation of an instrument needs to occur at a different angle than the angle of entry of the straight cannula/obturator combination. Therefore, there is a need for an inflexible cannula that can change the entry angle of an instrument at the surgical site of interest.

### SUMMARY OF THE INVENTION

According to the present invention there is provided a cannula, a straightener, and a cannula and straightener combination for insertion into tissue, the cannula including an elongated rigid hollow tube having a proximal end, a distal end, and a passageway extending therebetween. The distal end includes a memory of directionality to bend about a radius. The cannula straightener straightens the distal end of the cannula when inserted through the passageway of the cannula. The present invention also includes methods of inserting a cannula, removing a cannula, and performing surgery with a cannula in tissue or in fetal tissue. The present invention further includes methods of performing biliary cannulation in a transhepatic approach, performing fetal aortic valvuloplasty, placing catheters into the brachial plexus for pain management, placing catheters into the epidural space for anesthesia, thoracic dissection treatment, laparoscopic dissection, and hydro dissection of tissue.

### BRIEF DESCRIPTION ON THE DRAWINGS

Other advantages of the present invention will be readily appreciated as the same becomes better understood by reference to the following detailed description when considered in connection with the accompanying drawings wherein:

FIG. 1 is a side view of an introducer cannula with a curved distal end;

FIG. 2 is a side view of an introducer cannula and straightener combination;

FIG. 3 is a side view of one embodiment of a straightener;

FIG. 4 is a cross-sectional view taken along line A in FIG. 1 of the cannula showing a circular shape;

FIG. 5 is a cross-sectional view taken along line A in FIG. 1 of the cannula showing an oblong shape;

FIG. 6 is a cross-sectional view taken along line A in FIG. 1 of the cannula showing a square shape;

FIG. 7 is a partial view of a proximal end of a cannula showing an ergonomic knob;

FIG. 8 is a side view of a proximal end of a cannula with an indicator;

FIG. 9 is a perspective view of a proximal end of a cannula with an indicator;

FIG. 10 is a perspective view of a proximal end of a cannula with an indicator showing alignment with the bend of the distal end;

FIG. 11 is a side view of a proximal end of a cannula with an indicator of a light;

FIG. 12 is a side view of a proximal end of a cannula with an indicator of a computer screen;

FIG. 13 is a cross-sectional view taken along line B of FIG. 1 of the shape of the distal end;

FIG. 14 is a cross-sectional view of an operation in tissue where a cannula with a straightener of a magnet is bent by an outside magnet;

FIG. 15 is a partial view of a distal end of a cannula showing a leading edge;

FIG. 16 is a partial view of a straightener with a pointed mercedes tip;

FIG. 17 is a cross-sectional view taken along line C of FIG. 16 of the mercedes tip;

FIG. 18 is a partial view of a straightener with a blunt tip;

FIG. 19 is a cross-sectional view taken along line D of FIG. 2 of a straightener filling the passageway of a cannula;

FIG. 20 is a cross-sectional view taken along line D of FIG. 2 of a straightener partially filling the passageway of a cannula;

FIG. 21 is a cut-away view of a cannula with an instrument inside the passageway;

FIG. 22 is a cut-away view of a cannula with an instrument inside the passageway where the functional end of the instrument is at a different angle than the proximal end of the instrument;

FIG. 23 is a perspective view of a “D” shaped cross-section of a straightener;

FIG. 24 is a perspective view of a “U” shaped cross-section of a straightener;

FIG. 25 is a perspective view of an “I” shaped cross-section of a straightener;

FIG. 26 is a side view of an alternative embodiment of the combination of the present invention;

FIG. 27 is a side view of an alternative embodiment of the combination of the present invention;

FIG. 28 is a side view of an alternative embodiment of the combination of the present invention;

FIGS. 29A and 29B are side views of an alternative embodiment of the combination of the present invention;

FIGS. 30A and 30B are side views of an alternative embodiment of the combination of the present invention; and

FIGS. 31A and 31B are side views of an alternative embodiment of the combination of the present invention.

### DETAILED DESCRIPTION

Generally, the present invention provides a apparatus for use in performing minimally invasive surgery. More specifically, the present invention provides a cannula and cannula straightener combination for insertion into tissue. The cannula and cannula straightener combination is particularly useful in performing minimally invasive surgery where there is not easy access to a surgical site when inserting instruments in a straight direction.

A “cannula” refers to a surgical tube inserted into a body cavity, duct, or tissue to drain fluid, deliver medication, or allow surgery to be performed at a remote site by inserting instruments through the cannula. A cannula in this application is alternatively called an “introducer cannula,” and can be referred to by others by various names.

A “cannula straightener” is an elongated solid or hollow rod for insertion in a cannula. A cannula straightener is also referred to as a “straightener,” but can be called by others in various needed fields by various names. The straightener can be an obturator, a stylus, a trocar, or other similar device.

The term “tissue” means an aggregation of morphologically similar cells and associated intercellular matter acting together to perform one or more specific functions in the body. Four basic types of tissues include muscle, nerve, epidermal, and connective tissues. Tissue can refer to such specifics as vascular tissue, body cavity units, etc.

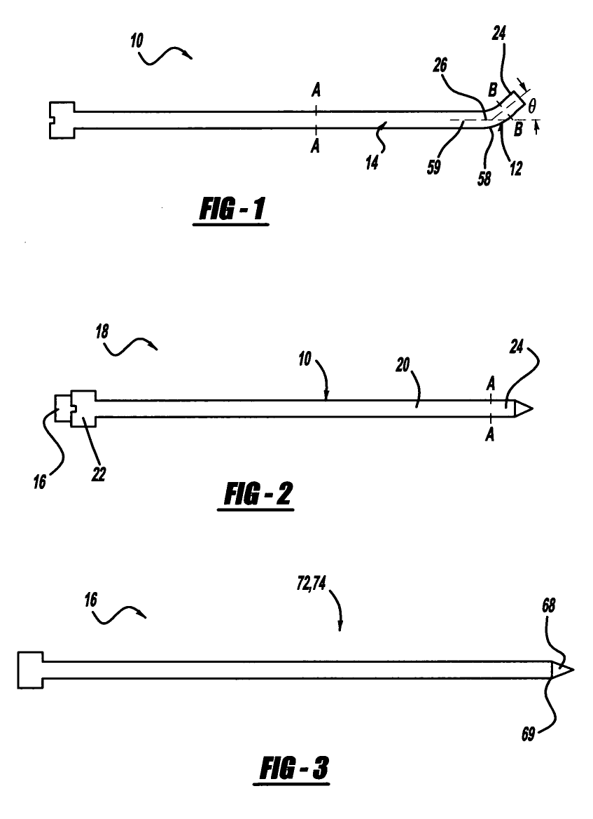

The cannula and cannula straightener combination of the present invention includes a cannula generally shown at 10 having a bendable arm 12 that maintains the structure and the functionality of a passageway 14 inside the cannula 10, as shown in FIG. 1. The passageway 14 has a generally round cross-sectional shape throughout its length. The functionality of the passageway 14 is to allow passage thereby of an insertion device or straightener or a medical instrument, as described below.

The cannula 10 is most often used with a cannula straightener 16. In FIG. 2, a cannula and cannula straightener combination 18 for insertion in tissue includes the cannula 10 being an elongated rigid hollow tube 20 having a proximal end 22, a distal end 24, and the passageway 14 extending therebetween. The distal end 24 of the cannula 10 includes a memory of directionality, as described below, to bend about a radius 26. A cannula straightener 16, shown alone in FIG. 3, acts to straighten the distal end 24 of the cannula 10 when inserted through the passageway 14 of the cannula 10 as described below.

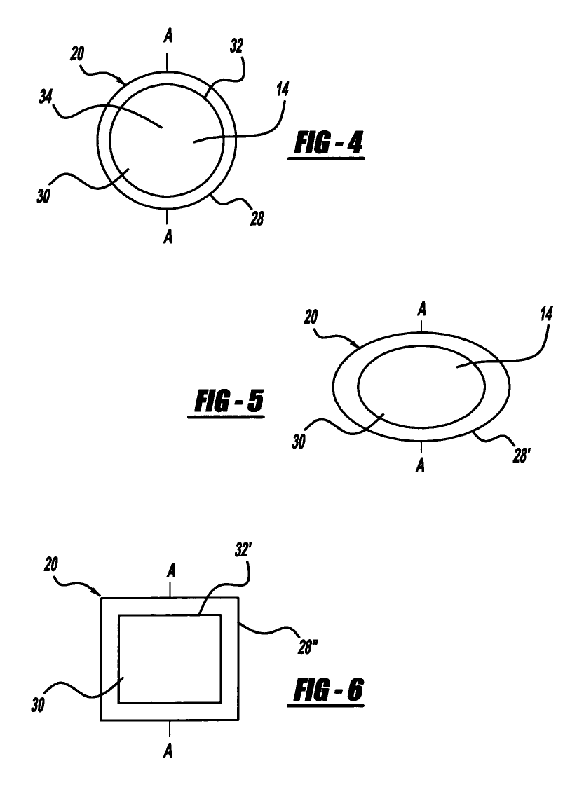

Preferably, the tube 20 has an outer circular cross-sectional shape 28 for smooth insertion into and through tissue. Other shapes can be used such as an oblong 28′, square 28″, or any other suitable shape as shown in FIGS. 4, 5, and 6.

The passageway 14 includes an inner surface 30 that can be any cross-sectional shape 32, such as circular 32 or square 32 as shown in FIGS. 4 and 6. The inner surface 30 can be custom designed to fit and guide a specific instrument 36 for insertion through the cannula 10. The inside diameter 34 of the passageway 14 can be any diameter appropriate for an instrument 36 to fit through in a surgical or other procedure. The cannula 10 can be of any suitable length to perform a specific procedure. The cannula 10 will likely be longer for use in fetal operations because it reaches through the tissue of the mother, through the womb, and through the fetal tissue to the surgical site. The cannula 10 can be all one piece, or alternatively, the proximal end 22 and distal end 24 can be fixably attached to the cannula tube 20.

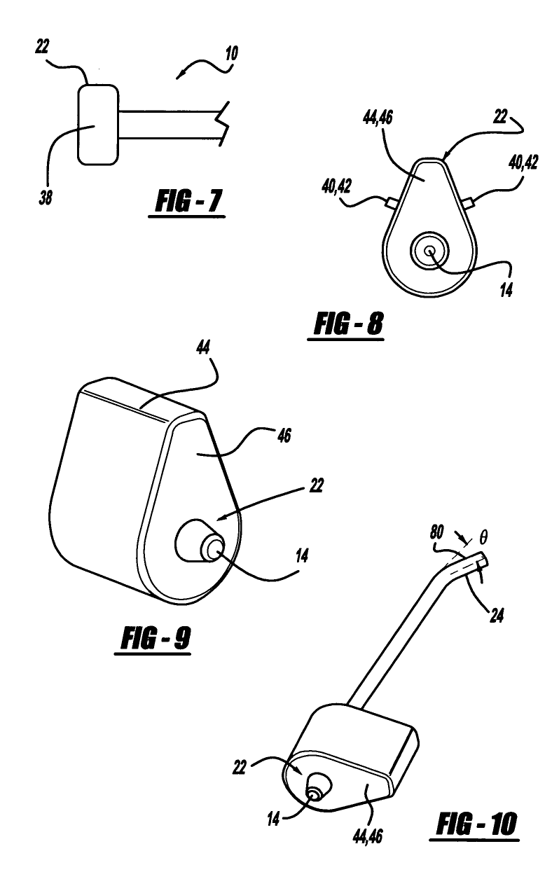

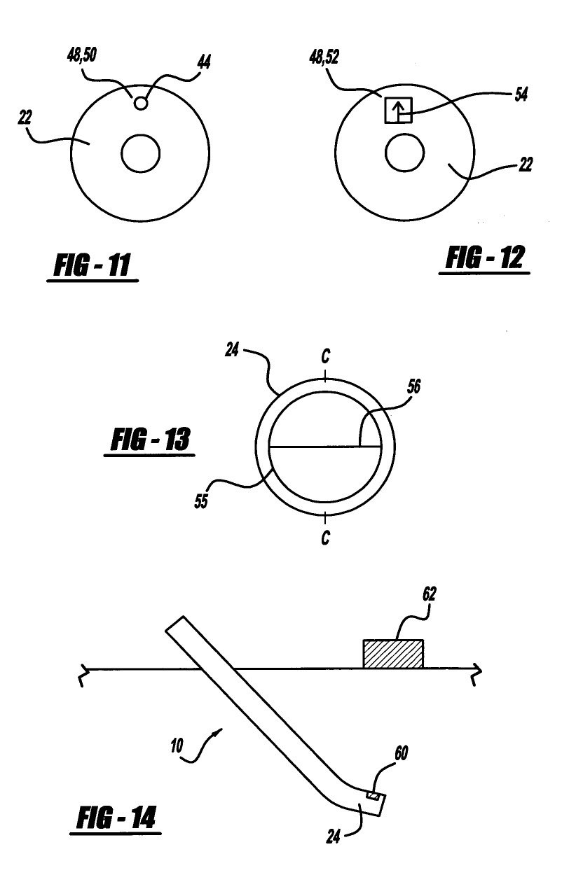

The proximal end 22 of the cannula 10, which is not inserted through tissue and will remain outside of the patient, can be any shape. For example, as shown in FIG. 7, the proximal end 22 can be an ergonomic knob 38 for the physician to hold onto during a surgical procedure. The proximal end 22 can also include valves 40 and other ports 42 for liquid and/or gas entering the cannula 10, for irrigation and/or suction, for sample collection, or for pressure transduction as shown in FIG. 8. Preferably, the proximal end 22 includes an indicator 44 for indicating the direction of the distal end 24 as shown in FIGS. 8, 9, and 10. The indicator 44 includes a signal 46 that allows the user to know the direction of the curve of the distal end 24. The indicator 44 allows for rotation of the cannula 10 to result in predictable redirection of its distal end 24 without angling or repositioning the entire cannula 10. As in FIGS. 8, 9, and 10, the indicator 44 includes elongated material in the direction of the bend 80 of the cannula 10. Alternatively, the direction of the distal end 24 can be indicated by an electronic device 48 such as a light 50 or on a computer screen 52, shown in FIGS. 11 and 12. For example, the indicator 44 could have an arrow 54 pointing in the direction of the bend 80. Any other suitable indicator 44 can be used.

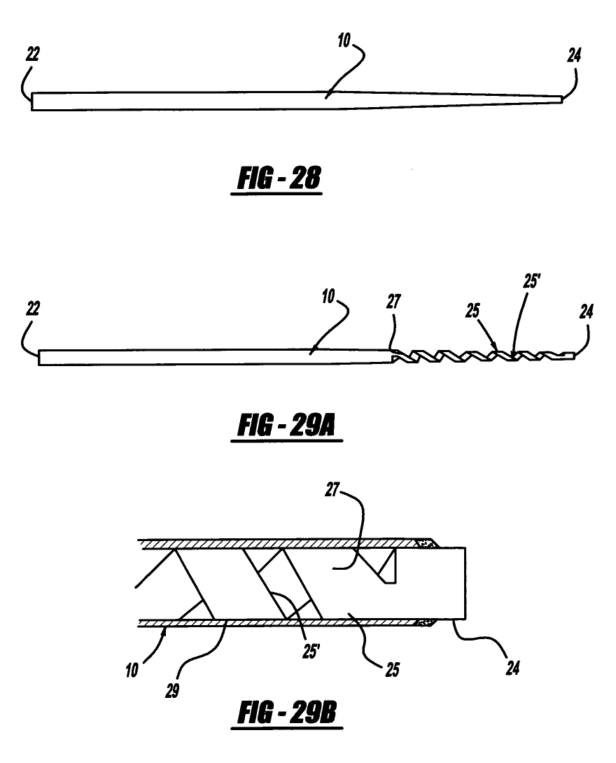

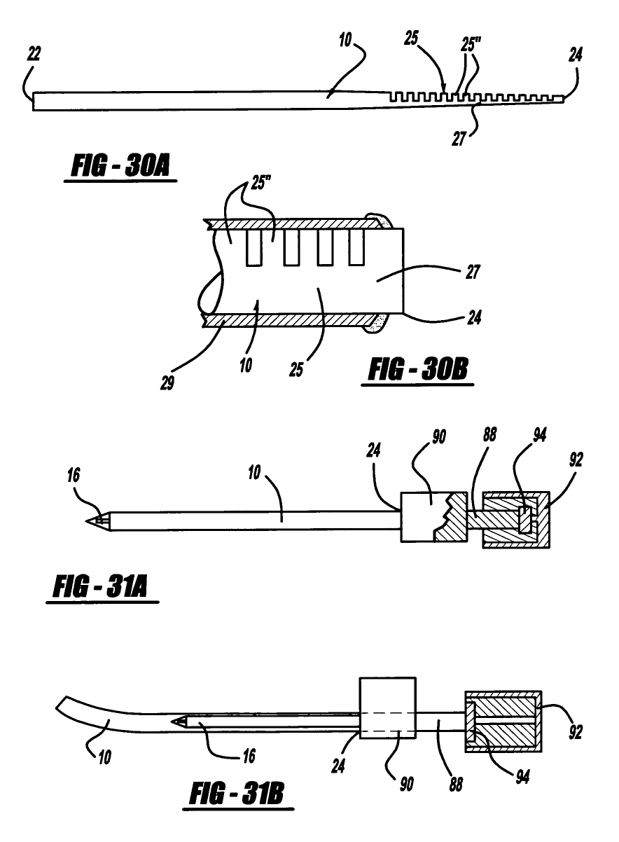

The distal end 24 of the cannula 10 is closest to the site of operation in the patient. The distal end 24 can be the same shape 55 and cross-sectional diameter 56 as the remainder of the cannula 10, shown in FIG. 13, or alternatively, it can be wider or narrower. Additionally, the distal end 24 can be tapered as shown in FIG. 28. The tapering limits grinding on the distal end 24 of the cannula 10 and the cannula 10 tapers as is approaches the distal end 24. The thickness of the cannula 10 can also be modified. For example, the cannula 10 at the proximal end 22 is preferably thicker than at the distal end 24. Specifically, the thickness at the proximal end 22 can be approximately 0.005 inches and the thickness at the distal end 24 can be approximately 0.002 inches. Further, the distal end 24 can include a textured portion 25, such as those shown in FIGS. 29A, 29B, 30A, and 30B. As shown in FIGS. 29A and 29B, the textured portion can include a spiral cut 25′ on the exterior surface 27 of the distal end 24. After creating the spiral cut 25′ on the distal end 24, the cannula 10 can be heat set for maintain the shape. Optionally, a tube 29 can be affixed about the exterior surface of the spirals 25′ on the distal end 24 to increase the stretch resistance. The tube 29 can be any biocompatible material that can be affixed to the cannula 10, examples of such materials are known to those of skill in the art. The preferred material is heat shrinkable. A preferred compound is a plastic such as heat shrink polyethylene terephthalate (PET). The texture portion 25 on the distal end 24 can also include slots 25″. The slots 25″ can be sized to allowed easier flexibility of the distal end 24. A tube 29 can be affixing to the exterior surface of the slots 25″ distal end 24 to increase the stretch resistance. The tube 29 can be any biocompatible material that can be affixed to the cannula 10, examples of such materials are known to those of skill in the art. The preferred material is heat shrinkable. Preferably, the distal end 24 is about 3 to 4 mm in length, however, the distal end 24 can be any suitable length. The length can be made specific to a procedure. The distal end 24 can bend about a radius 26, preferably from a memory of directionality, as shown in FIGS. 1 and 10. The distal end 24 bends at its juncture 58 with the cannula 10. The radius of bending can be any suitable angle theta, preferably 0 to 90 degrees from an axis 59 of the passageway 14, more preferably 0 to 60 degrees, and even more preferably 10 to 15 degrees from the axis 59 of the passageway 14. A memory of directionality allows the distal end 24 to return to the same angle theta each time a straightener 16 inside the cannula 10 is removed. The memory of directionality is a property of the material of the distal end 24.

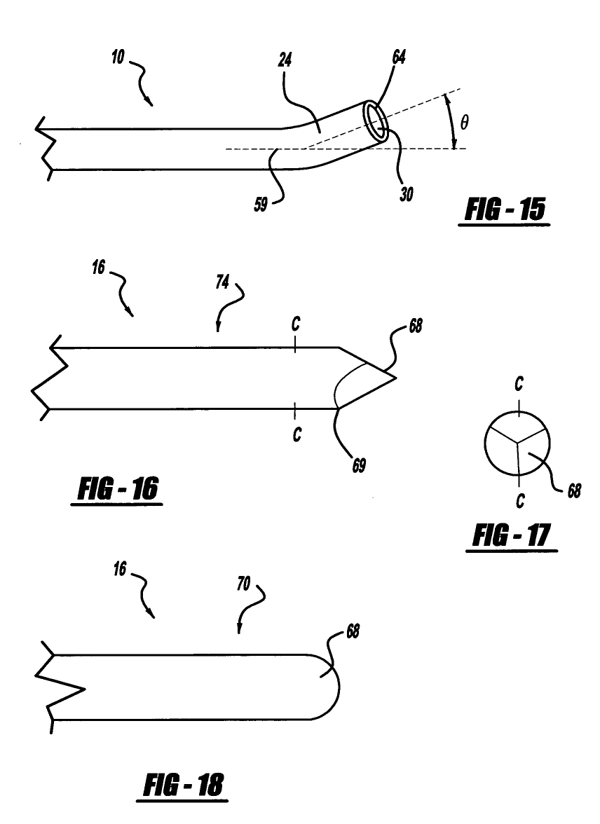

The distal end 24 can also be bent by alternative means. For example, a magnet 60 can be attached to a section of the distal end 24 and a physician can move a magnet of the opposite pole 62 over an area of the body to bend the distal end 24 towards the physician's magnet 62, as shown in FIG. 14.

During the bending of the distal end 24, the structural integrity and functionality of the passageway 14 in the cannula 10 is maintained. The cannula 10 does not kink so that an instrument 36 can fit through the passageway 14 without obstruction. The cannula 10 is rigid enough to withstand tissue pressure when inserted into and when inside a patient's tissue. The rigidness is maintained during rotation or maneuvering of the cannula 10 while in tissue, such as when maneuvering the distal end 24 after the straightener 16 is removed. The rigidness runs along the entire length of the cannula 10, including the distal end 24. Therefore, while the distal end 24 is flexible enough to bend around about a radius 26, it is also rigid enough to maintain the structural integrity and functionality of the passageway 14. This feature of the present invention is unlike many flexible plastic cannulas that kink and lose their passageway when a straightener is removed while inside tissue.

The distal end 24 further includes a leading edge 64 that is preferably tapered in toward inner surface 30 of the passageway 14, as shown in FIG. 15. The tapering allows the cannula 10 to move through tissue more easily, especially when the cannula 10 is in combination with the straightener 16. The distal end 24 can also be blunt.

A shape memory material can be used for the cannula 10, such as a shape memory polymer or other shape memory materials such as Nitinol. Preferably, a rigid shape memory material is used. In general, a shape memory material undergoes a change of crystal structure at its transformation temperature. Superelasticity, or pseudo elasticity, occurs when a material is in an environment that is above the temperature of its transformation temperature. The lower temperature crystal structure can be formed by applying stress to the material. Once sufficient stress is applied to the material above the transformation stress, the material undergoes deformation. Upon releasing the applied stress, the material returns to its original shape with no permanent deformation.

Preferably, the cannula 10 is made from Nitinol, which comes from a family of intermetallic materials that contain a nearly equal mixture of nickel (55 wt. %) and titanium. NITINOL is an acronym for Nickel Titanium Naval Ordnance Laboratory. Nitinol exhibits a unique phase transformation in the crystal structure when transitioning between the Austenite phase (high temperature, stronger state) and Martensite phase (low temperature, weaker state).

The behaviors shown in the phase transformation are commonly known as “Superelasticity” and “Shape Memory”. Superelasticity occurs when nitinol is mechanically deformed at a temperature above its Austenite Finish (Af) temperature. This deformation causes a stress-induced phase transformation from Austenite to Martensite. The stress-induced Martensite is unstable at temperatures above Af, and when the stress is removed, the material will immediately spring back to the Austenite phase and its pre-stressed position. Recoverable strains on the order of 8% are attainable. The high degree of elasticity, or “superelasticity”, is the most attractive property of nitinol and the most common aspect of the material in use today.

Shape Memory occurs when the nitinol is in its Martensitic phase and is deformed to a new shape. When the material is then heated above the Af temperature, it changes back to Austenite and the deformation is lost as the material returns to its pre-deformed, original shape. Up to 8% shape recovery is possible. Nitinol first was marketed for its thermal shape memory properties as pipe couplings, connectors and actuators. All nitinol exhibits both superelastic and shape memory behavior, but alloy composition and the material's thermo-mechanical processing history dictate the temperatures where these properties exist.

Any other material exhibiting shape memory behavior can also be used. For example, thermoplastic polymers can be used. A thermoplastic polymer can have one shape at room temperature, and transform into another shape at body temperature. The cannula 10 can also be made from other materials, such as a semi-flexible plastic, or a combination of plastic and metal, in other words, a combination of metallic and non-metallic materials. For example, the cannula 10 can be made of a stainless steel braid with a Teflon outer jacket. When the cannula 10 is not made from a material exhibiting shape memory behavior, the bend 80 at the distal end 24 can be accomplished in other ways. For example, a magnet 60 can be attached to a section of the distal end 24 as explained above. For any material used, the material should maintain the structural integrity and functionality of the passageway 14.

It is also desirable that the cannula 10 be imagable during an operation. The cannula 10 can be made with an imagable material so that the location of the cannula 10 in the patient's body can be determined by imaging methods such as ultrasound, magnetic resonance imaging (MRI), computed tomography (CT), X-ray, fluoroscopy, nuclear imaging or any other imaging method known in the art. In order for a cannula 10 to be imagable in an X-ray visualization procedure, the cannula 10 must be more absorptive of the X-rays than the surrounding tissues. Radiopaque materials are commonly used such as stainless steel and nickel-titanium alloys. Radiopaque markers can also be used. In MRI, polymers are typically used. Any other suitable imaging material can be used. The cannula 10 can be made of a combination of imagable materials and other biocompatible and/or shape memory materials. Methods of manufacturing the cannula 10 from the materials above are well known in the art.

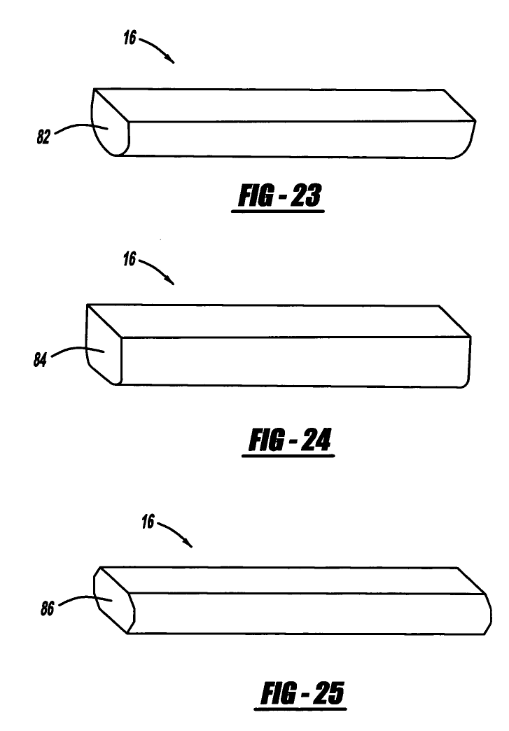

The cannula straightener 16 straightens the distal end 24 of the cannula 10 when inserted through the passageway 14 of the cannula 10. In general, the straightener 16 is a straight elongated rod 66 as shown in FIG. 3. The straightener 16 can also be any other suitable mechanism or device capable of straightening the cannula 10. The straightener 16 can take on a variety of cross-sectional shapes as shown in FIGS. 23, 24, and 25 such as a “D” shape 82, a “U” shape 84, or an “I” shape 86. The “D” shape is essentially a round rod with a flat portion. The “U” shape is essentially a square rod with rounded corners on one side. The “I” shape is essentially a round rod with flat portions on opposite sides. These shapes aid in preventing the pushing of debris back into the patient when the straightener 16 is inserted into the cannula 10. The straightener 16 can include a tip 68 on a distal end 69. The tip 68 can be any number of shapes. For example, the tip 68 can be pointed as shown in FIGS. 3 and 16. More specifically, FIGS. 16 and 17 shows a tip 68 with a pointed Mercedes tip 68. A pointed tip 68 is useful in making a sharp and less obtrusive puncture in tissue. The tip 68 can also be blunt, as shown in FIG. 18. Preferably, the tip 68 extends less than 1 mm beyond the leading edge 64 of the distal end 24 of the cannula 10. A short length of the tip 68 is desired because when the cannula and straightener combination 18 is positioned at the site of interest, and the straightener 16 is removed, the distal end 24 should not be far from the site. The straightener 16 can be hollow or solid.

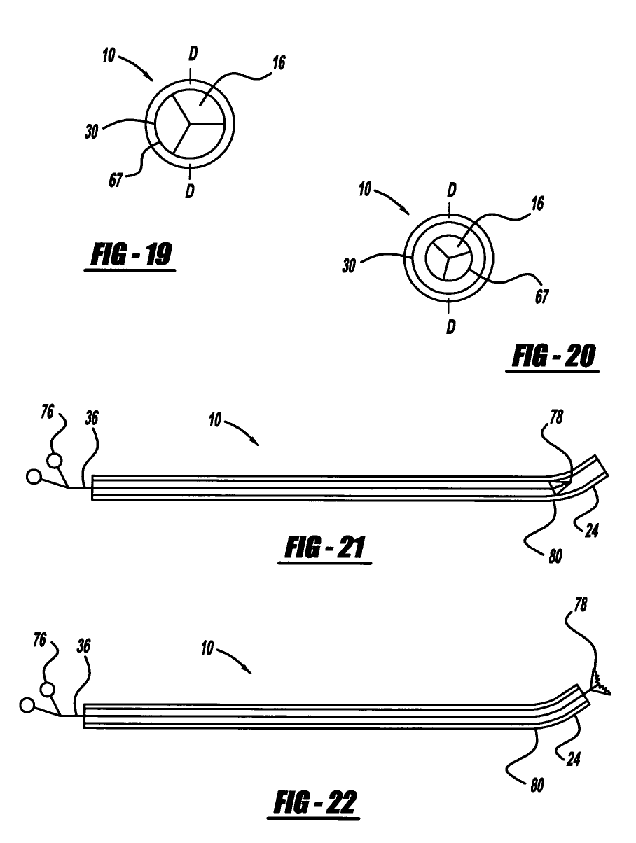

The straightener 16 can completely fill the passageway 14 as shown in FIG. 19. In other words, an outside surface 67 of the straightener 16 can be removably integral with the inner surface 30 of the passageway 14. A completely filled passageway 14 is ideal when it is desirable for no bodily fluids to escape up the passageway 14. Alternatively, as shown in FIG. 20, the outside surface 67 of the straightener 16 can be a distance from the inner surface 30 of the passageway so that the straightener 16 only partially fills the passageway 14.

The straightener 16 can be made from any suitable material. For example, the straightener 16 can be made of plastic or metal. The straightener 16 can be made of a material capable of being autoclaved for reuse in multiple procedures. Alternatively, the straightener 16 can be made of a disposable material. Methods of manufacturing the straightener 16 are well known in the art. The straightener 16 can be imagable in the same way as the cannula 10 as described above.

The straightener 16 can take on various forms and functions. For example, the straightener can be an obturator 70. An obturator 70 generally has a blunt tip 68, and is used when damage to surrounding delicate surface tissues is to be minimized. The straightener 16 can also be a stylus 72. The straightener 16 can also be a trocar 74. A trocar 74 generally includes a pointed tip 68 for puncturing tissue. The tip 68 of the straightener 16 can be distinguishable from the cannula 10 by the imaging methods described above. The straightener 16 can also be any other object or mechanism that allows for the distal end 24 to be straightened. For example, the straightener 16 can be a magnet 60 on the distal end 24 that is only activated to curve the distal end 24 in the presence of a magnet of the opposite pole 62.

In order to reduce friction between the cannula 10 and the straightener 16 either the surface of the cannula 10 or the straightener 16 can be made of a lubricious, non-galling material. One example of such a material includes, but is not limited to, nodular, thin, dense chrome (NTDC). Alternatively, as shown in FIGS. 31A and 31B, a junction 88 can be formed between a hub 90 on the proximal end 24 of the cannula 10 and a hub 92 on the proximal end 94 of the straightener 16. Preferably, the junction 88 is threaded thereby enabling more controlled motion between the cannula 10 and the straightener 16. The junction 88 is long enough to completely withdraw the straightener 16 through the cannula bend.

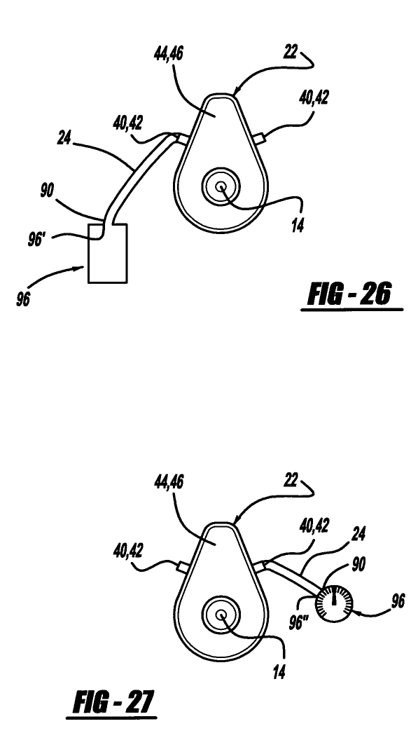

The cannula 10 can also include at least one attachment 96 that can be attached via a hub 90 on the proximal end 24 of the cannula 10 as shown in FIGS. 26 and 27. The attachment 96 can be any device capable of being affixed to the hub 90 of the cannula 10. Examples of such attachments 96 include, but are not limited to, a collection container 96′ and a manometer 96″.

As stated above, the attachment 96 can be a collection container 96′. The container 96′ is affixed to the hub 90 of the cannula 10. The container 96′ can include an aspirator, if necessary, for the removal of material via the cannula 10. The container 96′ enables samples to be collected from the individual in which the cannula 10 is placed. The sample is obtained through the passageway 14 of the cannula 10 using the distal end 24 of the cannula. In other words, the distal end 24 of the cannula 10 can be manipulated such that the distal end 24 is in contact or close proximity with the sample material. The sample material can then be extracted through the passageway 14 of the cannula 10 and collected in the container 96′.

Alternatively, the attachment 96 can be a manometer 96″, which is a device which measures pressure. The manometer 96″ can measure pressure wherever the distal end 24 of the cannula 10 is located.

In use, an introducer cannula 10 is inserted into a patient's tissue by inserting the straight tube 20 through tissue and curving a length of the tube at the bendable arm 12 when at a site of operation. This insertion is further defined by inserting the introducer cannula and cannula straightener combination 18 as described above into the tissue in a straight direction. The cannula 10 can be guided to the site of interest. The cannula 10 can further be guided by use of the indicator 44 on the proximal end 22 to indicate in which direction the cannula distal end 24 will curve. Insertion in a straight direction occurs because the distal end 24 of the cannula 10 is straightened by being in combination with the straightener 16. It would be very difficult or impossible to insert and guide the cannula 10 to the site of interest if the distal end 24 were curved. When in combination 18, the cannula 10 and the straightener 16 are straight in order to have exact control over the tip of the straightener.

When the cannula 10 is at the site of interest, a length of the cannula 10 is curved. The distal end 24 of the cannula 10 is bent about a radius 26. The bending of the distal end 24 can be accomplished by any of the methods as described above. The distal end 24 can be bent about any suitable radius 26 at any angle theta as described above. During the bending of the distal end 24, the passageway 14 is maintained inside the cannula 10. When the straightener 16 is a straight elongated rod 66 such as an obturator 70, stylus 72, or trocar 74, the straightener 16 is removed from the cannula 10 to allow the distal end 24 to return to its curved position. During removal of the straightener 16, the cannula 10 maintains the passageway 14 through its length.

Once the distal end 24 is curved at the site of interest, it can be slightly adjusted again by using the indicator 44. Adjustment should only be fine adjusting so as not to tear any adjacent tissues or structures.

An instrument 36 can be inserted through the passageway 14 as shown in FIGS. 21 and 22. Any instrument 36 can be inserted for the surgery or procedure of interest. Such instruments 36 are manipulated by a physician at a proximal end 76, and are functional at a distal end 78. The instrument 36 is able to curve around the bend 80 of the distal end 24 of the cannula 10, and therefore at least a portion of the instrument 36 is flexible. The functional end 78 of the instrument 36 is able to perform the desired function while curved about the radius 26 of the distal end 24. When the desired procedure is finished, the instrument 36 is removed from the cannula 10. During removal, the instrument distal end 78 curves back through the distal end 24 of the cannula 10. This process of inserting an instrument 36 and removing can be repeated to perform different procedures through the cannula 10.

The cannula 10 is removed when the procedure is finished. The cannula 10 can be removed as it is, i.e. in the curved distal end 24 position. The cannula 10 can be removed by straightening the distal end 24 of the cannula 10 about the radius 26 and removing the cannula 10 from tissue. The cannula 10 can also be removed by inserting a cannula straightener 16 as described above into the cannula 10, straightening the distal end 24 of the cannula 10 about a radius 26, and removing the cannula and cannula straightener combination 18 from tissue.

Surgery can be performed by inserting an introducer cannula 10 into tissue by any method as described above, introducing an instrument 36 through a passageway 14 of the cannula 10 while maintaining the bend 80 of the distal end 24 of the cannula 10 as described above, utilizing the instrument 36 to perform at least one step of a surgical procedure, removing the instrument 36 from the surgical site and from the cannula 10, and removing the introducer cannula 10 from the tissue by any method as described above.

There are many procedures of interest where it is desirable that a cannula 10 be inserted straight through tissue but then be able to change the directionality of the distal end 24 of the cannula 10 where an operative procedure is taking place. A curved distal end 24 of the cannula 10 is useful in reaching tissues and structures unreachable from a straight insertion of a cannula 10. The cannula 10 can also be used to manipulate the tissue. For example, in fetal uses, the cannula 10 (or several cannulae) can be inserted such that the distal end 24 of the cannula 10 is in contact with fetal tissue. The distal end 24 can then be manipulated in order to effectuate a change in position of the fetus within the uterus. The distal end 24 of the cannula can be advanced, redirected, and spun in order to effectuate the movement of the fetus or other tissue in need of such movement.

For example, the cannula 10 of the present invention can be used in operative procedures on a fetus. In this procedure, the introducer cannula and straightener combination 18 is inserted through the tissue of the mother (abdominal wall, naval, intravaginally), through the womb, and through the fetal tissue to the site of interest as described in the above methods. The distal end 24 of the cannula 10 is curved about a radius 26, and the passageway 14 is maintained according to the methods described above. An instrument 36 can be inserted in the passageway 14 to perform a surgical procedure of the operation. After removal of the instrument 36, another instrument 36 can be inserted or another step of the procedure can commence. The introducer cannula 10 can then be removed from the fetal tissue, womb, and mother's tissue by the methods described above.

The cannula 10 of the present invention is also useful for biliary cannulation in a transhepatic approach. Cannulation can be performed with small tapered catheters designed to guide wires or injections of contrast medium into biliary ducts. The need for biliary cannulation often occurs when there is an acute obstruction of the bile ducts, especially in patients with cholangitis. The obstruction can be a stone that has migrated down from the gallbladder. Patients with sepsis also can require drainage of the biliary tree. This can be accomplished by inserting a cannula 10 in a transhepatic approach.

The cannula 10 of the present invention is also useful for fetal aortic valvuloplasty. Neonatal aortic stenosis, narrowing of the aortal valve, is a serious, though treatable, congenital heart condition. Several different procedures are used in treating neonatal aortic stenosis, such as percutaneous, transvascular balloon valvuloplasty, in which the aortic valve orifice is dilated using a balloon catheter. When aortic stenosis presents in the second trimester fetus, it can develop into hypoplastic left heart syndrome, a condition that is fatal if untreated. Treatment with aortic valvuloplasty in the fetus may be advantageous. The cannula 10 can be used to place a balloon in the aortic valve during fetal balloon valvuloplasty.

The cannula 10 of the present invention is also useful for placing catheters into the brachial plexus for pain management. Often, injuries to the brachial plexus cause pain that can be debilitating for many years. Regional anesthesia can be used during an operation instead of general anesthesia. Catheters can be placed in the brachial plexus to make a continuous nerve block to manage acute pain. The cannula 10 can be used to introduce catheters into the brachial plexus.

The cannula 10 of the present invention is also useful for placing catheters into the epidural space for anesthesia. An epidural catheter can be placed through the skin into the epidural space of the spine by using the cannula 10. Catheters allow access to the epidural space for the administration of medication such as anesthetics. The catheters can be placed in the epidural space temporarily.

The cannula 10 of the present invention is also useful in treating thoracic aortic dissection. Thoracic aortic dissection is one of the most common traumas to the aorta. The essential feature is a tear in the intimal layer of the aorta, followed by formation and propagation of a subintimal hematoma. Several diseases affect the media of the aorta and make it prone to dissection, such as Marfan, Ehlers-Danlos, and other connective tissue diseases, and pulsatile flow and high blood pressure can contribute to the propagation of the dissection. The cannula 10 can aid in placing a graft on the damaged aorta, in replacing a defective valve in the aorta, or in any other surgical procedure needed in the aorta.

The cannula 10 of the present invention is further useful in laparoscopic dissection of tissue. Laparoscopic surgery is performed in the abdominal and pelvic regions. The cannula 10 can be used to introduce instruments 36 needed in the laparoscopic procedure such as a grasper or scissors at an angle to reach the surgical site.

The cannula 10 of the present invention is also useful in hydro dissection procedures of laparoscopic surgery. Hydro dissection uses the force of pulsatile irrigation with crystalloid solutions to separate tissue planes. The operating field is kept clear during the procedure. Hydro dissection is currently used in pelvic lymhadenectomy and pleurectomy. The cannula 10 can be used to introduce a hydro dissection sprayer at a certain angle to an operation site.

In any of these procedures, it is desirable to image the cannula 10 during the placement and removal, and also during the operative procedure itself. The cannula can be guided to and from the operative site by using an imaging method such as ultrasound, MRI, CT, X-ray, fluoroscopy, or nuclear imaging. Any other suitable imaging method can also be used.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used, is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

What is claimed:

1. A cannula and cannula straightener combination for insertion in tissue, comprising:
a cannula including an elongated rigid hollow tube having a proximal end, a distal end, and a passageway extending therebetween, said distal end having a memory of directionality to bend about a radius; and
cannula straightening means for straightening said distal end of said cannula when inserted through said passageway.

2. The combination of claim 1, wherein said cannula straightening means is chosen from the group consisting of an obturator, a stylus, and a trocar.

3. The combination of claim 1, wherein said distal end has a length from 3 to 4 mm.

4. The combination of claim 1, wherein said cannula straightening means further includes a straight shaft having a tip.

5. The combination of claim 4, wherein said tip has a shape selected from the group consisting essentially of blunt and pointed.

6. The combination of claim 4, wherein said tip leads said distal end of said cannula by less than 1 mm when said cannula straightening means is inserted through said passageway.

7. The combination of claim 4, wherein said tip is made with an imagable material.

8. The combination of claim 1, wherein said radius is further defined as an angle from 0 to 90 degrees.

9. The combination of claim 1, wherein said proximal end includes indicator means for indicating the direction of said distal end.

10. The combination of claim 1, wherein said cannula is made from a material selected from the group consisting essentially of a shape memory material and an imagable material.

11. The combination of claim 1, wherein said distal end further includes a tapered leading edge.

12. The combination of claim 1, wherein an outside surface of said cannula straightening means is integral with an inside surface of said passageway.

13. The combination of claim 1, wherein said cannula straightening means has a diameter smaller than a diameter of said passageway.

14. The combination of claim 1, wherein said cannula is of a plastic.

15. The combination of claim 1, wherein said cannula includes a magnetic portion on said distal end.

16. The combination of claim 1, wherein said straightener includes a cross-sectional area chosen from the group consisting of a D shape, a U shape, or an I shape.

17. The combination of claim 1, wherein said distal end of said cannula includes a textured portion.

18. The combination of claim 1, wherein said textured portion is textured in a manner selected from the group consisting essentially of tapering, spiral cutting, and slotted cutting.

19. The combination of claim 1, wherein said combination includes junction means between said proximal end of said cannula and a proximal end of said cannula straightening means for connecting said cannula and said cannula straightening means in a controllable manner.

20. A cannula for insertion in tissue, comprising:
an elongated hollow tube having a proximal end, a distal end, and a passageway extending therebetween, said distal end having a memory of directionality to bend about a radius and maintaining said passageway when bent.

21. The cannula of claim 20, wherein said radius is further defined as an angle from 0 to 60 degrees.

22. The cannula of claim 20, wherein said proximal end includes indicator means for indicating the direction of said distal end.

23. The cannula of claim 20, wherein said cannula is made from a material selected from the group consisting essentially of a shape memory material and an imagable material.

24. The cannula of claim 20, wherein said distal end has a length from 3 to 4 mm.

25. The cannula of claim 20, wherein said distal end further includes a tapered leading edge.

26. A cannula for insertion in tissue including a bendable arm that maintains a passageway inside said cannula.

27. The cannula of claim 26, wherein said cannula is made from a plastic.

28. The cannula of claim 26, wherein said cannula includes a magnetic portion on said distal end.

29. A cannula and instrument combination, comprising:
a cannula including an elongated hollow tube having a proximal end, a distal end, and a passageway extending therebetween, said distal end having a memory of directionality to bend about a radius; and
an instrument for performing surgical operations, said instrument extending along a length of said passageway, said instrument including a functional end able to slide around said bend and function outside of said passageway at a site of operation.

30. A method of inserting an introducer cannula, including the steps of:
inserting a straight tube through tissue; and
curving a length of said tube when disposed at a site of operation.

31. The method of claim 30, further defining the method as:
inserting through tissue in a straight direction an introducer cannula and cannula straightener combination comprising a cannula including an elongated hollow tube having a proximal end, a distal end, and a passageway extending therebetween, said distal end having a memory of directionality to bend about a radius, and cannula straightening means for straightening the distal end of the cannula;
bending the distal end of the cannula about a radius; and
removing the straightener from the cannula, wherein said cannula retains a hollow center.

32. The method of claim 31, further including the step of guiding the introducer cannula to an operation site after said inserting step.

33. The method of claim 31, further including the step of introducing an instrument through the cannula, wherein the distal end of the cannula remains bent about a radius.

34. The method of claim 31, further including the step of adjusting the position of the cannula by an indicator on the proximal end of the cannula after said bending step.

35. The method of claim 30, further comprising the step of imaging the cannula.

36. The method of claim 31, further defining said bending step as bending the distal end from 0 to 60 degrees about the radius.

37. A method of removing a cannula, including the steps of:
straightening a distal end of the introducer cannula about a radius; and
removing the introducer cannula from tissue.

38. The method of claim 37, further including the step of inserting a cannula straightening means into an introducer cannula before said straightening step, and further defining said removing step as removing the introducer cannula and straightening means combination from tissue.

39. A method of performing surgery, including the steps of:
inserting an introducer cannula into tissue by inserting a straight tube through tissue and curving a length of said tube when disposed at a site of operation;
introducing an instrument through the cannula, wherein the distal end of the cannula remains bent about a radius;
utilizing the instrument to perform at least one step of a surgical procedure;
removing the instrument from the site and from the cannula; and
removing the introducer cannula from tissue by straightening a distal end of the introducer cannula about a radius and removing the introducer cannula from tissue.

40. The method of claim 39, further including the step of removing the cannula straightening means from the cannula after the inserting step, wherein the cannula retains a hollow center.

41. The method of claim 39, for use performing a surgery selected from the group consisting essentially of biliary cannulation in a transhepatic approach, fetal aortic valvuloplasty, placing catheters into the brachial plexus for pain management, placing catheters into the epidural space for anesthesia, thoracic dissection treatment, laparoscopic dissection, and hydro dissection of tissue.
